(19) United States
(12) Patent Application Publication
(10) Pub. No.: US 2025/0345114 A1
(43) Pub. Date: Nov. 13, 2025

(54) Ablation catheter arrangement and device for the ablation of tissue

(71) Applicant: Stockert GmbH, Freiburg im Breisgau (DE)

(72) Inventors: Fabian Kees, Freiburg im Breisgau (DE); Fritz Koch, Freiburg (DE)

(21) Appl. No.: 19/182,936

(22) Filed: Apr. 18, 2025

(30) Foreign Application Priority Data
May 8, 2024 (DE) 102024112952.2

(51) Int. Cl. A61B 18/14; A61B 18/00; A61B 18/12

(52) U.S. Cl. CPC A61B 18/1492; A61B 18/1206; A61B 2018/00267; A61B 2018/00577; A61B 2018/00642; A61B 2018/00672; A61B 2018/00678; A61B 2018/00791; A61B 2018/1407

(57) ABSTRACT

The present invention relates to an ablation catheter arrangement and to a device for the ablation of tissue. The ablation catheter arrangement includes an inner catheter module having a first shaft and a first flexible outer structure fastened to the first shaft and being configured to assume a collapsed state and an expanded state. The ablation catheter arrangement also includes an outer catheter module having a second shaft and a second flexible outer structure fastened to the second shaft and being configured to assume an initial state and an end state. The second shaft is configured to receive an inner catheter module in the second shaft so that the second flexible outer structure assumes the initial state when the first flexible outer structure is in the collapsed state, and the second flexible outer structure assumes the end state when the first flexible outer structure is in the expanded state.

### CROSS-REFERENCE TO RELATED APPLICATIONS

This application claims priority to German Patent Application Serial No. DE 10 2024 112 952.2 filed May 8, 2024.

### BACKGROUND

### Field

The present invention relates to an ablation catheter arrangement and to a device for the ablation of tissue.

### Discussion of the Related Art

In recent years, the treatment of tissue by means of pulsed electrical fields has become established as an increasingly relevant clinical technique. The use of short high-voltage pulses and the high electrical field strengths associated therewith, which act on the tissue, have, however, already been the subject of intensive research for more than four decades. This application method is categorised as a non-thermal procedure, since it is based on the delivery of short pulses with a high voltage amplitude, which generate between active electrode pairs a locally strong electrical field in the region of up to several hundred volts per centimetre. This field strength leads to the formation of pores in the cell membranes. If the electrical field exceeds a specific threshold value, which is required for the formation of pores in the lipid bilayers of the cell membranes, and if the tissue is exposed to that field over a critical period of time, the electroporation becomes irreversible. The pores remain permanently open, which ultimately leads to programmed cell death (apoptosis) of the cell in question.

Pulsed field ablation has proved to be a promising treatment method inter alia for the treatment of cardiac arrhythmias. Cardiac arrhythmias, such as, for example, atrial fibrillation, occur when diseased regions of the heart tissue form or transmit abnormal electrical signals. These disrupt the normal heartbeat and cause an asynchronous rhythm. A possible form of treatment for such arrhythmias is the interventional suppression of the electrical signal transduction of the disturbing signals. By selective ablation of the cardiac tissue by means of the supply of energy via a catheter, a non-conducting lesion can be formed, which prevents the propagation of undesirable electrical signals. In the case of a specific type of arrhythmia, atrial fibrillation, the cause of the disturbing electrical signals lies in the pulmonary veins, which lead into the left atrium of the heart. By means of catheter ablation (pulmonary vein isolation), the pulmonary veins are purposively isolated electrically from the left atrium for treatment.

Renal denervation is a method in which increased sympathetic activities are suppressed in order to treat high blood pressure or other cardiovascular disorders and chronic diseases of the kidneys. This is effected via a minimally invasive procedure in which, with the aid of a special catheter, the nerve lines around the renal arteries are suppressed. The catheter is inserted into the renal artery, and the nerves running outside are generally cauterised through the vessel wall by means of thermal or chemical ablation.

A number of clinical studies have already shown that a combined treatment comprising pulmonary vein isolation and renal denervation can lead to a reduction in the recurrence of atrial fibrillation following ablation.

In order to carry out the combined treatment comprising pulmonary vein isolation and renal denervation, it is necessary to carry out treatment in different regions of the body of a patient, in particular in different organs and/or vessels. Separate devices are conventionally used for this purpose. The same or similar problems exist when other treatment methods are combined. Here too, different devices often have to be used in order to combine the treatment methods.

Document U.S. Pat. No. 10,016,233 B2 describes a system for the combined treatment of atrial fibrillation by cardiac ablation and renal denervation. The ablation is carried out with the aid of conventional ablation methods. Two independent catheters are additionally necessary for carrying out the cardiac ablation and the renal denervation.

Document U.S. Pat. No. 10,517,672 B2 describes a system which comprises a high-voltage generator and a balloon catheter system and which is able to deliver IRE pulses via that system. This takes place at the inner wall of the renal arteries, in order to ablate and cauterise the external nerve fibres through the wall.

However, there continues to be a need for an arrangement and a system which can flexibly be used in the combination of multiple treatment methods. In particular, there is a need for catheters which can flexibly be used in different treatment methods.

### SUMMARY

According to a first aspect, an ablation catheter arrangement is proposed. The ablation catheter arrangement has at least one inner catheter module. The ablation catheter arrangement has an outer catheter module. The at least one inner catheter module has (in each case) at least one first shaft and a first flexible outer structure fastened to the first shaft. The first flexible outer structure is configured to assume a collapsed state and an expanded state. The outer catheter module has a second shaft and a second flexible outer structure fastened to the second shaft. The second flexible outer structure is configured to assume an initial state and an end state. The second shaft (of the outer catheter module) is configured to receive an inner catheter module of the at least one inner catheter module in the second shaft. The second shaft is configured to receive an inner catheter module of the at least one inner catheter module in the second shaft in such a manner that the second flexible outer structure assumes the initial state when the first flexible outer structure is in the collapsed state, and the second flexible outer structure assumes the end state when the first flexible outer structure is in the expanded state.

The initial state may also be referred to as the initial form state, and the end state may also be referred to as the end form state, since both states can relate to or denote a form of the outer catheter module, in particular of the second flexible outer structure of the outer catheter module. The at least one inner catheter module and the outer catheter module can be of different types or relate to different catheter types. The at least one inner catheter module and the outer catheter module can have a different outer structure, for example. One or more, in particular each, of the at least one inner catheter module can be of the same type or relate to the same catheter type. However, one or more, in particular each, of the at least one inner catheter module can differ from one another, for example in terms of dimensioning and/or in terms of size and/or in terms of their expanded state. There can be one or more intermediate states between the initial state and the end state. The intermediate states of the second flexible outer structure can be assumed in accordance with a degree of expansion of the first flexible outer structure.

The at least one inner catheter module can be based on at least one inner catheter or correspond to at least one inner catheter. The outer catheter module can be based on at least one outer catheter or correspond to at least one outer catheter. At least the outer catheter module can be based on an ablation catheter or correspond to an ablation catheter.

The at least one inner catheter module can be configured as a balloon catheter module or have a balloon catheter module. The balloon catheter module can be based on a balloon catheter or correspond to a balloon catheter. The first flexible outer structure can be configured as a flexible membrane or have a flexible membrane. In this case, the flexible membrane can assume a tubular form, for example, in the collapsed state. In the expanded state, the flexible membrane can assume a balloon-shaped form, for example. In the one intermediate state or the plurality of intermediate states, the flexible membrane can assume a balloon-like or balloon-shaped form. For example, starting from the initial state, the intermediate states can assume an increasingly balloon-shaped form up to the end state.

The inner catheter module can have a proximal end and a distal end. The first flexible outer structure, in particular the flexible membrane, can be arranged between the distal end and the proximal end. The outer catheter module can have a proximal end and a distal end. The second flexible outer structure can be arranged between the distal end and the proximal end.

A plurality of electrodes can be arranged on an outer side of the second flexible outer structure. The second flexible outer structure can have a plurality of ribs or be formed by a plurality of ribs. At least one electrode can be arranged or formed on each of the plurality of ribs.

The second flexible outer structure can be basket-shaped, for example. For example, the plurality of ribs can extend in a basket shape or form a basket shape. For example, the plurality of ribs can extend from a proximal region of the outer catheter module, in particular of the second shaft, to a distal region of the outer catheter module, in particular of the second shaft.

An inner shaft can be arranged and configured in the second shaft in such a manner as to receive in each case an inner catheter module of the at least one inner catheter module. The dimensions of the inner shaft, in particular a cross section of an interior of the inner shaft and/or a length of the interior, can be such that one of the at least one inner catheter module can be received in the inner shaft. For example, a plurality of inner catheter modules can be provided as the at least one inner catheter module. In this case, in each case an inner catheter module of the plurality of inner catheter modules can be capable of being inserted into the inner shaft of the outer catheter module. If the inserted inner catheter module, in particular the first flexible outer structure thereof, is brought from its collapsed state into its expanded state, the outer catheter module, in particular the second flexible outer structure thereof, assumes an end state that is dependent on the expanded state or that corresponds at least approximately to the expanded state. If a different inner catheter module of the plurality of inner catheter modules is then introduced into the inner shaft and assumes its expanded state, which is different from the preceding expanded state, then the outer catheter module assumes an end state that is dependent on the expanded state or that corresponds at least approximately to the expanded state and that is thus different from the preceding end state. In this manner, different end states, in particular form states or forms, can be assumed with the same ablation catheter arrangement. Thus, by introducing different inner catheter modules, different end states for the outer catheter module, in particular for the second flexible outer structure thereof, can be effected. Likewise, by introducing different inner catheter modules, different intermediate states for the outer catheter module, in particular for the second flexible outer structure thereof, can be effected.

At a distal end of the outer catheter module there can be arranged a distal electrode. At a proximal end of the outer catheter module there can be arranged a proximal electrode. The distal electrode and/or the proximal electrode can be of annular form. Thus, the outer catheter module can have, proximally and distally to the second flexible outer structure, in each case an annular electrode attached to or arranged on the second shaft.

According to a second aspect, a device for the ablation of a tissue of a patient is proposed. The device has an ablation catheter arrangement according to the first aspect. The device has a signal generator arrangement, which is connected or can be connected to the ablation catheter arrangement. The device has a control and evaluation unit, which is connected or can be connected to the ablation catheter arrangement and/or the signal generator arrangement.

The control and evaluation unit can be adapted to carry out and/or to give instructions about and/or to control different measurements and/or treatment processes. For example, the control and evaluation unit can be configured to carry out and/or to give instructions for and/or to control a tissue impedance determination and/or an ablation and/or a denervation measurement and/or a denervation. The control and evaluation unit can, for example, be configured to carry out a tissue impedance determination and, for example subsequently, an ablation. In addition or alternatively, the control and evaluation unit can be configured to carry out a denervation measurement and, for example subsequently, a denervation.

The signal generator arrangement can have a first signal generator for generating a radiofrequency (RF) signal and a second signal generator for generating a signal for a pulsed field ablation (PFA). The RF signal can be used for a conventional thermal ablation. The signal for the PFA (=PFA signal) can be used for an irreversible electroporation.

The control and evaluation unit can be adapted to deliver a first electrical signal into the tissue via an electrode constellation, in particular the ablation catheter arrangement. The control and evaluation unit can be adapted to deliver a first electrical signal into the tissue and to receive a second electrical signal from the tissue via the electrode constellation, in particular the ablation catheter arrangement. The electrode constellation can be understood as being a pairwise association of electrodes. The signal generator-evaluation unit can be adapted to actuate electrode pairs and/or electrode constellations and/or to switch between electrodes (electrode pairs) and/or electrode constellations.

An electrode constellation can be flexibly defined by/formed by/composed of the proximal and/or the distal electrode and/or one or more of the plurality of electrodes of the ablation catheter arrangement that are arranged on the second flexible outer structure. Any desired electrode constellations can be formed, depending on the field of use.

Depending on the field of use, the first electrical signal can be configured as an electrical current signal and the second electrical signal can be configured as an electrical voltage signal, or vice versa. For example, the control and evaluation unit can be adapted to determine at least one, in particular local, tissue impedance from the electrical current signal transmitted into the tissue (=the first electrical signal) and the electrical voltage signal received from the tissue (=second electrical signal). The control and evaluation unit can be adapted to determine at least two, in particular local, tissue impedances by means of at least two electrode constellations/electrode pairs/electrodes (to be actuated), in particular chosen/switched in (temporal) succession.

The control and evaluation unit can be configured to control/actuate the first signal generator and/or the second signal generator to deliver a signal. In this way, the RF signal and/or PFA signal can be delivered as required.

The control and evaluation unit can be configured to actuate the first signal generator or the second signal generator to deliver a signal, for example an RF signal, in dependence on at least one electrode temperature. For example, the control and evaluation unit can be configured to actuate the first signal generator if the electrode temperature falls below a temperature limit value and to actuate the second signal generator to deliver a signal, for example a PFA signal, if the electrode temperature assumes or exceeds the temperature limit value.

Further features, properties, advantages and possible modifications will become clear to a person skilled in the art from the following descriptions, in which reference is made to the accompanying drawings.

### BRIEF DESCRIPTION OF THE DRAWINGS

FIG. 1 is a schematic representation of a bipolar IRE pulse according to an exemplary embodiment.

FIG. 2 is a schematic representation of a pulse protocol having multiple series or bursts of bipolar pulses according to an exemplary embodiment.

FIG. 3 shows a schematic representation of a pulse protocol for a combined treatment with RF and pulsed field ablation having at least one series of RF signals and at least one series or burst of bipolar IRE pulses according to an exemplary embodiment.

FIG. 4 shows a schematic representation of an outer catheter module of an ablation catheter arrangement in collapsed form.

FIG. 5 shows a schematic representation of an inner catheter module in expanded form.

FIG. 6 shows a schematic representation of an ablation catheter arrangement according to an exemplary embodiment having the outer catheter module from FIG. 4 and the inner catheter module from FIG. 5.

FIG. 7 shows a schematic representation of an inner catheter module in expanded form.

FIG. 8 shows a schematic representation of an ablation catheter arrangement according to an exemplary embodiment having the outer catheter module from FIG. 4 and the inner catheter module from FIG. 7.

FIG. 9 shows a schematic representation of an outer catheter module according to a variant of the catheter module from FIG. 4 in collapsed form.

FIG. 10 shows a schematic representation of an ablation catheter arrangement according to an exemplary embodiment having the outer catheter module from FIG. 9 and the inner catheter module from FIG. 7.

FIG. 11 shows schematic connections of electrodes of the ablation catheter arrangement from FIG. 6 for local impedance measurement.

FIG. 12 shows schematic connections of electrodes of the ablation catheter arrangement from FIG. 6 for the stimulation and measurement of nerve activity.

FIG. 13 shows a flow diagram of method steps for carrying out a denervation.

### DETAILED DESCRIPTION OF THE EMBODIMENTS

Irreversible electroporation (IRE) is primarily a non-thermal procedure, which uses only a small amount of electrical energy and thus effects an increase in the tissue temperature by only a few° C. This distinguishes it significantly from conventional RF ablation (RF: radiofrequency), in which the tissue temperature increases by 20 to 70° C. and cells are destroyed by heat. In IRE, bipolar pulses are generally used, that is to say a combination of positive and negative electrical pulses, in order to avoid as far as possible muscle contractions, which usually occur in the case of the application of DC voltage. These pulses can be applied between two bipolar electrodes of a catheter or between a catheter electrode and a body surface electrode, which is usually applied to the patient's back.

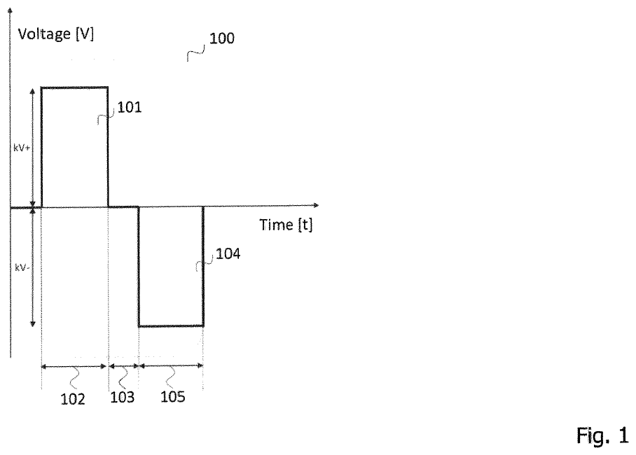

FIG. 1 is a schematic representation of a biphasic IRE pulse according to an exemplary embodiment. It shows the voltage V of the biphasic PFA pulse 100 as a function of the time t in an IRE ablation procedure. The present exemplary embodiments relate to an IRE generator which is configured as a voltage source. Consequently, the IRE signals are here described in the form of their voltages. The biphasic IRE pulse comprises a positive pulse 101 and a negative pulse 104, the terms “positive” and “negative” referring to an independently chosen polarity of two electrodes that are actuated for the ablation and between which the biphasic pulse is applied. The amplitude of the positive pulse 101 is denoted kV+ and lasts for time 102. Analogously, the amplitude of the negative pulse 104 is denoted kV− and has a temporal width 105. Between the two pulse phases 101 and 104 there can be a delay time 103. Both the two temporal pulse widths 102 and 105 and the amplitudes kV+ and kV− can be configured independently of one another and can therefore vary in an exemplary embodiment.

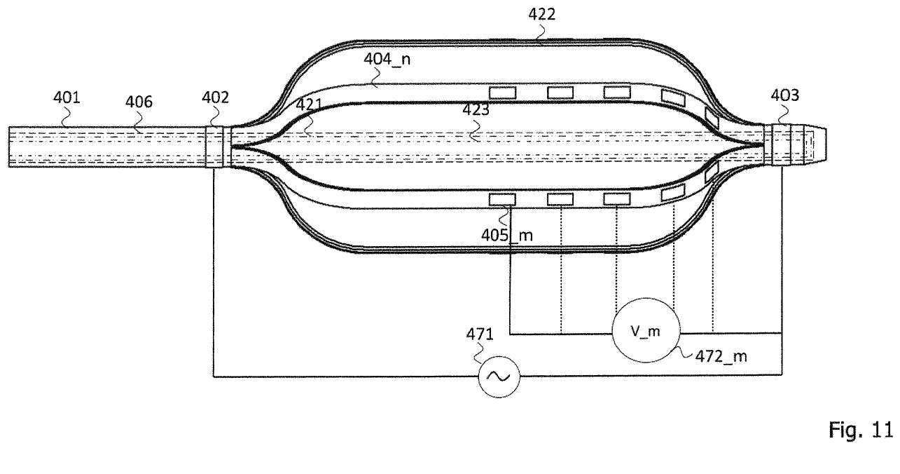

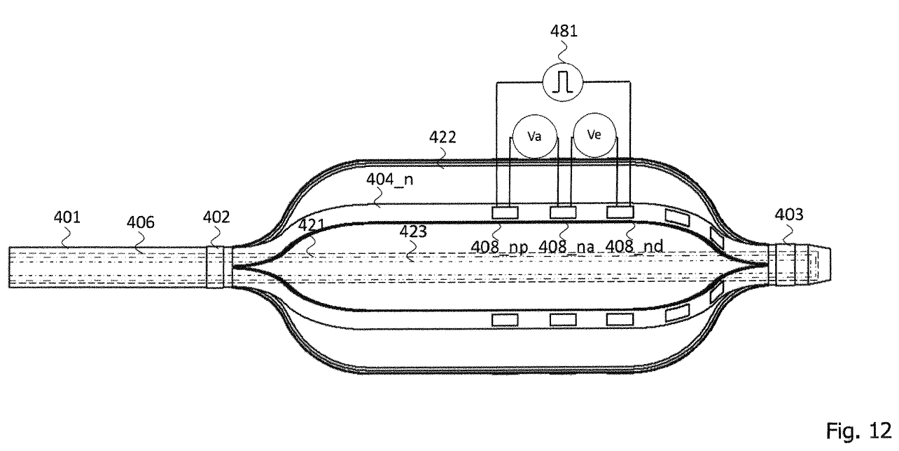

The bipolar pulse 100 shown schematically in FIG. 1 can be generated by the signal generator arrangement. The formats, which determine the properties of the bipolar pulse 100, can be predefined by a user. The values of the pulse deflection kV+, kV− of the positive pulse 101 and of the negative pulse 104 can be, for example, +500 KV. The third time interval 103 between the positive pulse 101 and the negative pulse 104 can be, for example, 2.5 μs. The pulse width 102 of the positive pulse 101 can differ from the pulse width 105 of the negative pulse 104. The difference in the pulse widths is not shown in FIG. 1. The pulse that is generated can be used as will be explained hereinbelow inter alia with reference to FIGS. 11 and 12.

In order that the IRE pulses generate pores in the tissue, the electrical field strength E, defined by the pulses, at the tissue between a pair of at least two electrodes must exceed a tissue-dependent threshold value Eth. For example, the threshold value for cardiac cells is approximately 500 V/cm, while for bones it is 3000 V/cm. These differences in the threshold field strengths allow the selective application of IRE in different tissues. In order to achieve the required field strength, the voltage to be applied to an electrode pair depends both on the target tissue and also on the distance between the electrodes and on the size of the electrodes themselves. These parameters likewise also influence the thermal energy input during the ablation and thus the temperature peaks which can occur at the tissue to be treated. The applied voltages can reach several kilovolts, which is substantially higher than the voltages of 10-200 V that are typical in the case of thermal RF ablation.

The bipolar pulsed field ablation pulse (bipolar PFA pulse) for IRE comprises a positive and a negative pulse (as shown by way of example in FIG. 1), which are applied between two electrodes with a pulse width of from 1 to 5 μs and an interval between the positive and negative pulses of from 1 to 5 μs. The bipolar pulses are combined to form pulse sequences, wherein each sequence can comprise over one hundred bipolar pulses with a pulse-to-pulse interval of from 1 to 10 ms. The pulse sequences in each case form a burst, wherein the entire pulse packet of the IRE ablation is composed of from 1 to 20 bursts/burst units, each of which has a burst-to-burst interval of from 1 to 1000 ms. The total duration of an ablation can be up to 10 s.

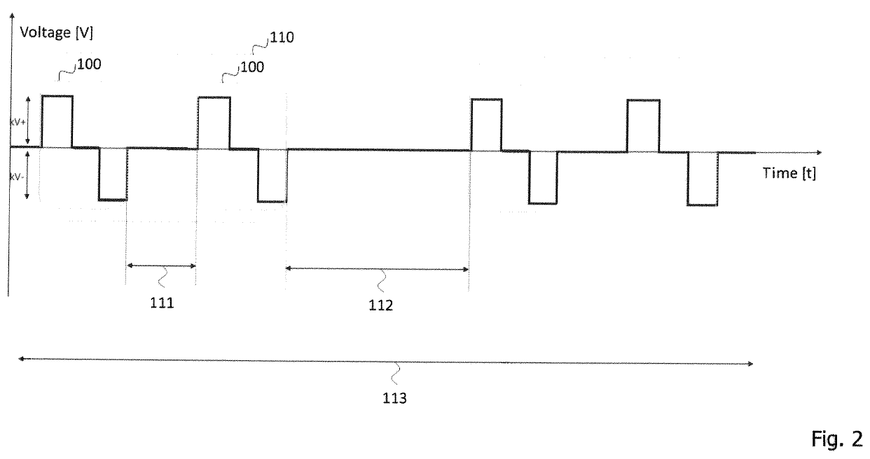

FIG. 2 is a schematic representation of a pulse protocol having a plurality of bursts of biphasic pulses according to an exemplary embodiment. The pulses 100 are delivered in the form of one or more bursts or pulse packets 110 over the duration of the entire IRE procedure 113. Each burst 110 comprises a defined number N of biphasic pulses 100, the pulses being separated by a time interval 111. There is again a delay time 112 between the delivery of the individual bursts 110.

The pulse protocol shown in FIG. 2 can form an electrical signal, which can be generated and used as described in greater detail hereinbelow. The electrical signal, as mentioned, is configured as a burst-signal sequence. Two bursts can be seen, one of which is provided with the reference numeral 110. Each burst has at least two bipolar pulses 100. Each bipolar pulse 100 that occurs in the burst-signal sequence has, for example, the properties of the format from FIG. 1. The first number of bursts, here by way of example two bursts, the number of bipolar pulses, the second time interval 111, and the first time interval 112 between two successive bursts 110 can be defined by a user. The burst-signal sequence to be seen extends over a duration 113, which corresponds to the duration of the irreversible electroporation.

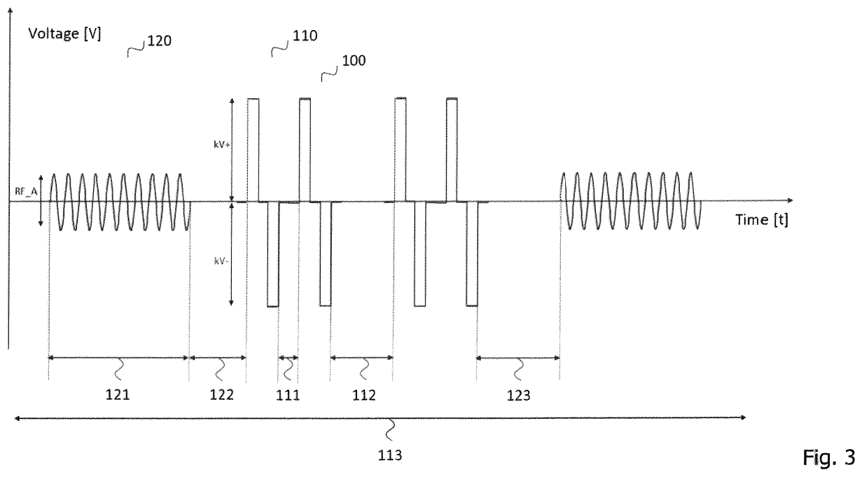

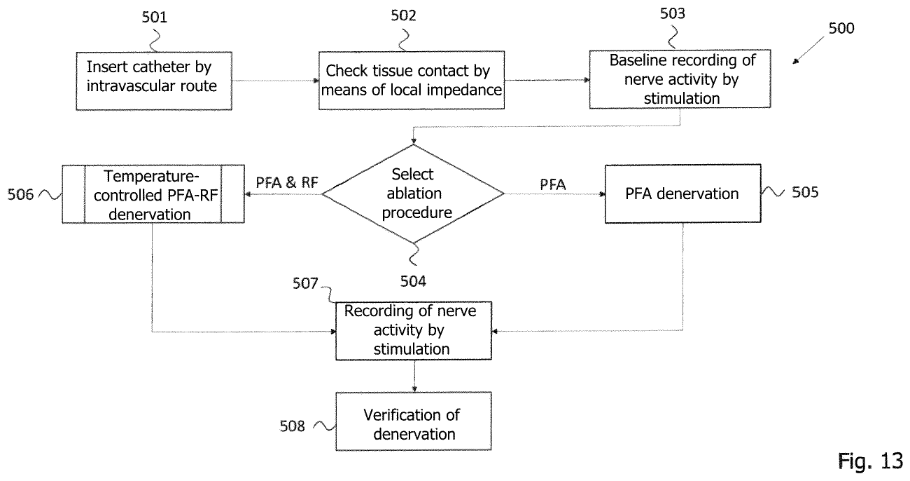

FIG. 3 shows a schematic representation of a procedure protocol having at least one burst of biphasic IRE pulses (PFA pulses) combined with at least one RF energy burst 120 according to an exemplary embodiment, as can be used herein. An example of a use will be described in relation to FIG. 13. The RF energy and the IRE pulses are delivered in the form of one or more bursts 120 and 110 over the duration of the entire combined procedure 113. Each IRE burst comprises a defined number N of bipolar pulses 100, the pulses being separated by a time interval 111. The RF burst is described by a sinusoidal signal of amplitude RF_A and duration 121. An RF burst is followed by a delay time 122. Both the duration of the RF burst and the subsequent delay time can be controlled on the basis of the instantaneously measured temperature at the ablation electrodes. There is a delay time 112 between the delivery of the individual IRE bursts 110. There is a delay time 123 between an IRE burst and a further delivery of the RF burst.

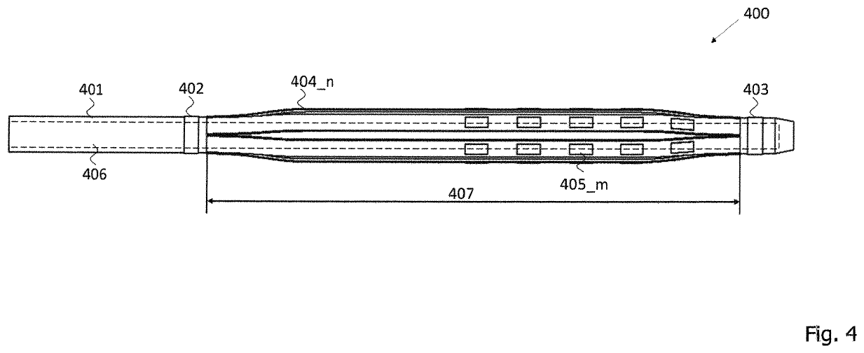

FIG. 4 shows an embodiment of an outer catheter module 400 of an ablation catheter arrangement in the collapsed state, which is used for manoeuvring into and out of the organ/vessel of the patient. The catheter module 400 has an outer, optionally manoeuvrable, shaft 401 for insertion into an organ/vessel of the patient, and an inner shaft 406 for insertion of an inner catheter module of the ablation catheter arrangement. The outer shaft 401 is designed such that it is divided over a defined length 407 and forms a number n of ribs/splines 404_n. A defined number m of electrodes 405_m is attached to each of these ribs/splines 404_n. Proximally and also distally to the ribs/splines 404_n there is in each case an annular electrode 402 and 403, said electrodes being fixedly connected to the outer shaft 401. All the electrodes 405_m, 402 and 403 are exposed to an external environment and are electrically connected via one or more electrical lines, which extend from the proximal end over the shaft 401 to the electrodes. The electrical supply lines are covered in such a manner that they are electrically insulated both from one another and from the external environment.

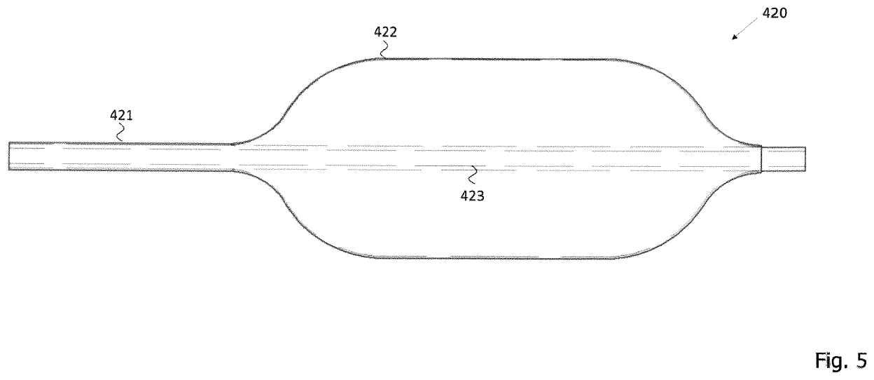

FIG. 5 shows an embodiment of an inner catheter module 420 of the ablation catheter arrangement in an expanded state, which can be used to shape the outer catheter module 400 during a procedure. The inner catheter module 420 has an outer shaft 421, an inner shaft 423, and an expanded balloon membrane 422 fastened to the outer shaft 423. Expansion takes place via the inner shaft 421, which has dedicated holes to the balloon membrane 422.

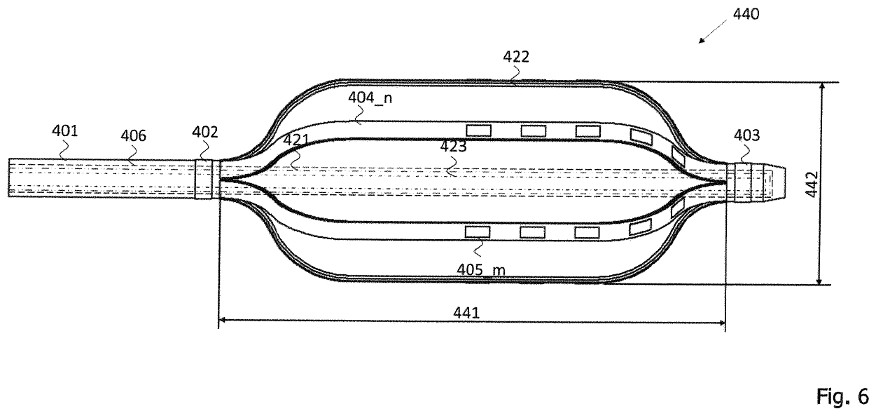

FIG. 6 shows an embodiment of an ablation catheter arrangement 440, in particular of a catheter system 440 forming a coherent unit, having the expanded balloon element from FIG. 5 as the inner catheter module and having the outer catheter module from FIG. 4, as can be used during a procedure. The inner catheter module 420 has been inserted axially into the inner shaft 406 of the outer catheter module 400, so that the balloon element and the ribs/splines 404_n are flush with one another. By expansion of the balloon membrane 422, the shape of a basket, formed by the ribs/splines 404_n, of the outer catheter module 400, together with the electrodes 405_m attached thereto, thus conforms to the defined shape of the balloon element 422. This imparts an effective diameter 442 and an effective length 441 to the outer catheter module 400. These two parameters are adjustable specifically via the balloon module and are defined by the procedure to be carried out and the geometry of the organ/vessel to be treated. The embodiment shown in FIG. 6 is an example of the performance of a renal denervation.

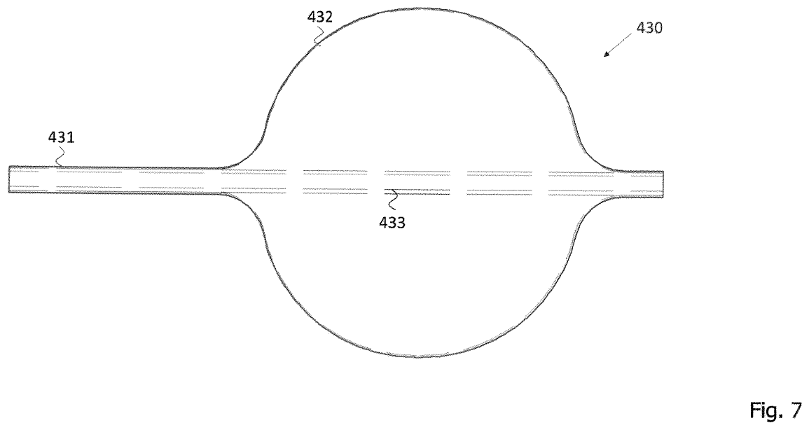

FIG. 7 shows a further embodiment of an inner catheter module 430 of the ablation catheter arrangement/catheter system in the expanded state, which can be used to shape an outer catheter module during a procedure. The inner catheter module 430 has an outer shaft 431, an inner shaft 433, and an expandable balloon membrane 432 fastened to the outer shaft. Expansion takes place via the inner shaft, which has dedicated holes to the balloon membrane 432.

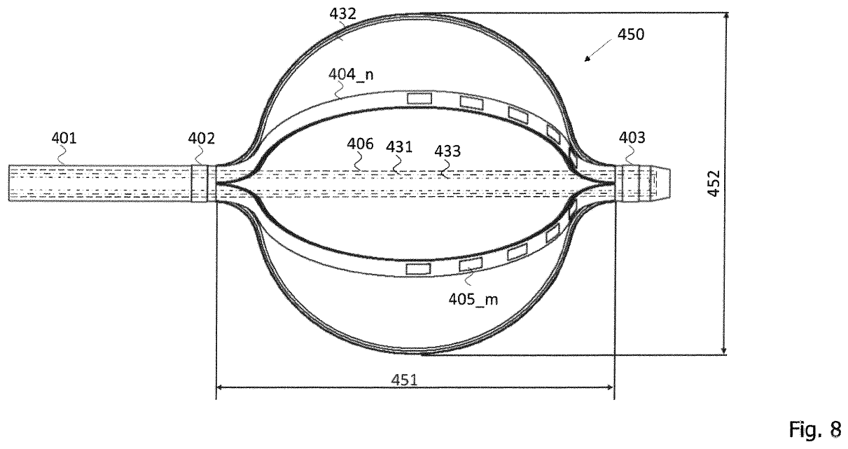

FIG. 8 shows a further embodiment of an ablation catheter arrangement 450, in particular of a catheter system forming a coherent unit, having an inner catheter module 430 with the expanded balloon element from FIG. 7 and an outer catheter module 400 from FIG. 4, as can be used during a procedure. The inner catheter module 430 has been inserted axially into the inner shaft 406 of the outer catheter module 400, so that the balloon element and the ribs/splines 404_n are flush with one another. By expansion of the balloon membrane 432, the shape of the basket formed by the ribs/splines 404_n, together with the electrodes 405_m attached thereto, thus conforms to the defined shape of the balloon element. This imparts an effective diameter 452 and an effective length 451 to the outer catheter module 400. These two parameters are adjustable specifically via the balloon module and are defined by the procedure to be carried out and the provided geometry of the organ/vessel to be treated. The embodiment shown in FIG. 8 is an example of the performance of a pulmonary vein isolation.

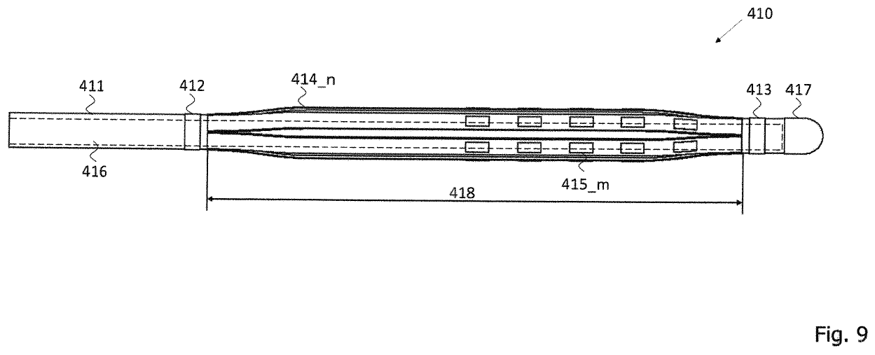

FIG. 9 shows a further embodiment of an outer catheter module 410 of the ablation catheter arrangement in a collapsed state, which is used for manoeuvring into and out of the organ/vessel of the patient. The outer catheter module 410 has an outer, optionally manoeuvrable, shaft 411 for insertion into an organ/vessel of the patient, and an inner shaft 416 for insertion of the inner catheter module of the arrangement. The outer shaft 411 is designed such that it is divided over a defined length 418 and forms a number n of ribs/splines 414_n. A defined number m of electrodes 415_m is attached to each of these splines 414_n. Proximally and distally to the ribs/splines 414_n there is in each case an annular electrode 412 and 413, said electrodes being fixedly connected to the outer shaft 411. In addition, a further electrode 417 is attached to the distal tip of the shaft 411, said further electrode having an atraumatic shape. All the electrodes are exposed to an external environment and are electrically connected via one or more electrical lines, which extend from the proximal end over the shaft to the electrodes. The number of electrical supply lines are covered in such a manner that they are electrically insulated both from one another and from the external environment.

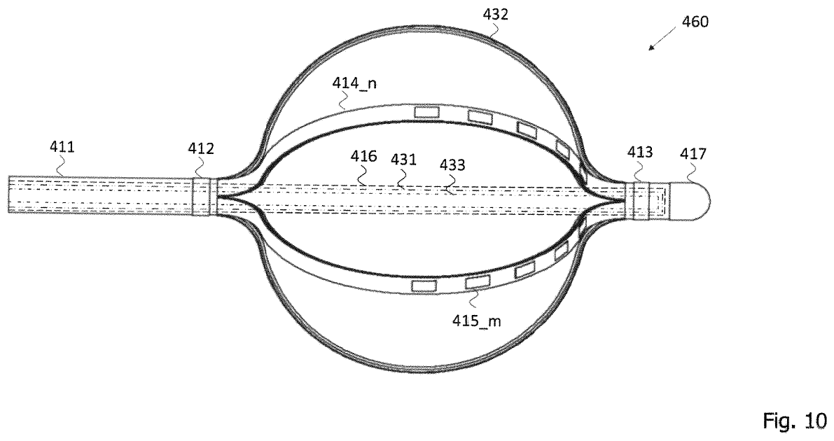

FIG. 10 shows a further embodiment of an ablation catheter arrangement 460, in particular of a catheter system forming a coherent unit, having an inner catheter module 470 with the expanded balloon element from FIG. 7 and an outer catheter module 410 from FIG. 9, as can be used during a procedure. The embodiment of FIG. 10 is a variant of the embodiment of FIG. 8 and varies compared to the form shown in FIG. 8 only by the use of an outer catheter module 410 that in this embodiment corresponds to that from FIG. 9, that is to say a design with an additional tip electrode 417.

FIG. 11 shows, by way of example for all the embodiments of the ablation catheter arrangement that are shown, a mechanism and an actuation for determining the local impedance of the target tissue. An electrical current 471 is applied between the proximal ring electrode 402 and the distal ring electrode 403. In order to determine the impedance with the aid of Ohm's law, the voltages 472_m from each of the ablation electrodes 405_m of each individual rib/spline 404_n to the distal ring electrode 403 are measured. This corresponds to the total number of spline electrodes 405_m over the number n of ribs/splines 404_n, so that the same number of impedance metrics is correspondingly obtained and the properties of the target tissue can be determined very selectively.

FIG. 12 shows, schematically, a mechanism and actuation for determining the nerve conductivity. This is measured once prior to the ablation as the starting value and again following the procedure, in order to characterise the denervation. In the schematic actuation depicted here, a distinction is made between the measurement of afferent nerves, which lead from the kidney to the central nervous system, and efferent nerves, which lead from the central nervous system to the kidney. An electrical stimulation pulse 481 is applied between the electrodes arranged proximally 408_np and distally 408_nd to the ablation electrode 408_na. In parallel, the resulting voltage Va from the ablation electrode to the electrode arranged proximal thereto is measured, on the one hand, in order to characterise the denervation of the afferent nerves. On the other hand, the resulting voltage Ve from the ablation electrode to the electrode arranged distal thereto is measured, in order to characterise the denervation of the efferent nerves. This mechanism happens independently at each of the ribs/splines n, so that the same number of efferent and afferent voltages are obtained in total.

FIG. 13 shows a schematic sequence 500 for carrying out a denervation procedure. In the first step 501, the ablation catheter arrangement is inserted into the patient by the intravascular route. Once the ablation catheter arrangement is in position, it is checked, by measuring the local impedance at the ablation electrodes of the plurality of splines, whether the electrodes have sufficiently good contact with the tissue (step 502). If this contact is not ensured for individual electrodes, they can selectively be switched off by the control and evaluation unit. In the next step 503, the starting value of the afferent and efferent nerve conductivity is recorded by the mechanisms described in FIG. 12. Then, in step 504, it is possible to choose between two types of procedure: a denervation by means of pulsed field ablation (PFA) or a combination procedure of PFA and RF energy. In the case of PFA denervation (step 505), the procedure is carried out on the basis of an exemplary protocol as described in FIG. 2 by means of biphasic IRE pulses. If a PFA-RF combination procedure (step 506) is chosen, the procedure is carried out on the basis of a combination protocol as described by way of example in FIG. 3. Irrespective of the chosen denervation procedure, the nerve activity is subsequently recorded again (step 507) in order to characterise the denervation (step 508) as described in FIG. 12.

What is claimed is:

1. An ablation catheter arrangement comprising:
at least one inner catheter module having a first shaft and a first flexible outer structure fastened to the first shaft, said first flexible outer structure being configured to assume a collapsed state and an expanded state; and
an outer catheter module having a second shaft and a second flexible outer structure fastened to the second shaft, said second flexible outer structure being configured to assume an initial state and an end state, wherein the second shaft is configured to receive an inner catheter module of the at least one inner catheter module in the second shaft in such a manner that the second flexible outer structure assumes the initial state when the first flexible outer structure is in the collapsed state, and the second flexible outer structure assumes the end state when the first flexible outer structure is in the expanded state.

2. The ablation catheter arrangement according to claim 1, wherein the at least one inner catheter module is configured as a balloon catheter module or includes a balloon catheter module, and the first flexible outer structure is configured as a flexible membrane or includes a flexible membrane.

3. The ablation catheter arrangement according to claim 1, wherein a plurality of electrodes are arranged on an outer side of the second flexible outer structure.

4. The ablation catheter arrangement according to claim 1, wherein the second flexible outer structure includes a plurality of ribs or is formed by a plurality of ribs.

5. The ablation catheter arrangement according to claim 4, wherein at least one electrode is arranged or formed on each of the plurality of ribs.

6. The ablation catheter arrangement according to claim 1, wherein the second flexible outer structure is basket-shaped.

7. The ablation catheter arrangement according to claim 1, wherein an inner shaft is arranged and configured in the second shaft in such a manner as to receive an inner catheter module of the at least one inner catheter module.

8. The ablation catheter arrangement according to claim 1, wherein the outer catheter module:
comprises, proximally to the second flexible outer structure, an electrode attached to or arranged on the second shaft; and/or
comprises, distally to the second flexible outer structure, an electrode attached to or arranged on the second shaft.

9. The ablation catheter arrangement according to claim 8, wherein the electrodes are annular electrodes.

10. A device for the ablation of a tissue of a patient, comprising:
an ablation catheter arrangement according to claim 1;
a signal generator arrangement connected to or can be connected to the ablation catheter arrangement; and
a control and evaluation unit connected to or can be connected to the ablation catheter arrangement and/or the signal generator arrangement.

11. The device according to claim 10, wherein the signal generator arrangement includes a first signal generator for generating a radio frequency (RF) signal and a second signal generator for generating a signal for a pulsed field ablation.

12. The device according to claim 11, wherein the control and evaluation unit is configured to actuate the first signal generator and/or the second signal generator to deliver a signal.

13. The device according to claim 12, wherein the control and evaluation unit is configured to actuate the first signal generator and/or the second signal generator to deliver the signal in dependence on at least one electrode temperature.

14. The device according to claim 13, wherein the control and evaluation unit is configured to actuate the first signal generator to deliver the signal if the electrode temperature falls below a temperature limit value and to actuate the second signal generator to deliver the signal if the electrode temperature assumes or exceeds the temperature limit value.
